(19) United States
(12) Patent Application Publication
(10) Pub. No.: US 2012/0244107 A1
(43) Pub. Date: Sep. 27, 2012

(54) BIODEGRADABLE HYDROGEL

(75) Inventors: Heike Heckroth, Odenthal (DE); Christoph Eggert, Koln (DE); Hartmut Nefzger, Pulheim (DE); Jörg Hofmann, Krefeld (DE); Edward Browne, Koln (DE); Klaus Lorenz, Dormagen (DE)

(73) Assignee: Bayer MaterialScience AG, Leverkusen (DE)

(21) Appl. No.: 13/502,397

(22) PCT Filed: Oct. 7, 2010
(86) PCT No.: PCT/EP10/06125

§ 371 Date: May 14, 2012

(30) Foreign Application Priority Data
Oct. 21, 2009 (EP) 09013258.0

(51) Int. Cl. A61K 31/785

(52) U.S. Cl. 424/78.37

(57) ABSTRACT

The present invention relates to a hydrogel on the basis of polyurethane or polyurethane urea, said hydrogel comprising hydrolysable functional groups in the polymer chain, to a method for producing the hydrogel and to the use of the hydrogel as adhesion barriers.

The present invention relates to a hydrogel based on polyurethane or polyurethaneurea, a process for preparing the hydrogel and the use of the hydrogel as an adhesion barrier.

Adhesions are among the most frequent complications after interventions in the abdominal and pelvic region. Adhesions are fibrous bands which generally form within the first seven days after an operation, in the course of the healing process. They cause tissues and organs which are normally separated from one another to grow together, which can give rise to a multiplicity of complications such as, for example, chronic pain, infertility or a life-threatening intestinal occlusion. Products able to reduce the formation of adhesions have been developed in recent years to avoid such complications.

Hydrogels have been used as adhesion barriers as well as other materials. Hydrogels are water-containing polymers whose chains are linked covalently to form a three-dimensional network. These networks swell in water up to an equilibrium volume with substantial shape retention. Network formation, although predominantly due to chemical linking together of individual polymer chains, is also possible physically through electrostatic, hydrophobic or dipole-dipole interactions between individual segments of polymer chains. Desired properties of hydrogels are specifically targetable via the choice of monomers used for polymer construction, the type of crosslinking and the crosslink density.

Hydrogels are typically based on poly(meth)acrylic acids, poly(meth)acrylates, polyurethanes, polyvinylpyrrolidone or polyvinyl alcohol. They are generally highly compatible with living tissue and therefore are often proposed for use as adhesion barriers.

Polyurethane hydrogels from hydrophilic NCO prepolymers are known per se. They are used for the medical treatment of wounds and as primary wound dressings for example. They have the advantage of keeping specifically dry wounds moist in a controlled manner, which is beneficial for wound healing.

DE 10 2006 050 793 describes polyurethane hydrogels based on aliphatic NCO polyether prepolymers. The hydrogels are also used as adhesion barriers. However, the systems described biodegrade only very slowly in the body, if at all. Degradation generally takes more than six months. Yet an adhesion barrier should degrade within a few months, since they are merely meant to protect the organs temporarily from growing together during the wound healing process.

The problem addressed by the invention was therefore that of preparing a biocompatible adhesion barrier that is biodegraded over a period of less than 6 months without the degradation products formed having any cell and tissue toxicity.

This problem is solved by a hydrogel based on polyurethane or polyurethaneurea, having hydrolyzable functional groups in the polymer chain and being obtainable by reaction of
A) polyisocyanate prepolymers having the hydrolyzable groups in the polymer chain,
B) water
C) optionally hydroxyl-amino compounds having at least one tertiary amino group and at least three hydroxyl groups,
D) optionally catalysts, and
E) optionally auxiliary and addition agents,
where said polyisocyanate prepolymers A) are obtainable by reaction of
A1) polyisocyanates with
A2) polyols having the hydrolyzable groups in the polymer chain,

characterized in that said polyols A2) are polyesters and/or polyetheresters that are liquid at room temperature and have a DIN 53019 shear viscosity at 23° C. in the range from 200 to 8000 mPas and preferably in the range from 400 to 4000 mPas.

A hydrolyzable group for the purposes of the invention is a group which, under physiological conditions in man and mammals, are splittable into at least two mutually separate sub-groups during an average period of less than 6 months.

The hydrogels of the present invention are biocompatible, i.e., neither they themselves nor their degradation products have any cell or tissue toxicity. In addition, they are biodegraded in less than 6 months.

The specific polyetheresters and/or polyesters used according to the present invention are notable for their ease of processing.

The polyetherester polyols and/or the polyesters may have a hydroxyl number of 20 to 140 mg KOH/g, preferably of 20 to 100 mg KOH/g and/or an acid number of 0.05 to 10 mg KOH/g, preferably of 0.1 to 3 mg KOH/g and more preferably of 0.15 to 2.5 mg KOH/g.

Polyols A2) may preferably have an average OH functionality of 2 to 4.

Preferably, the hydrolyzable functional groups are ester, acetal and/or carbonate groups.

The preparation of suitable polyester polyols is described in EP 2 095 832 A1 for example.

Polyetherester synthesis can also utilize mixtures of higher molecular weight and lower molecular weight polyols.

Such (in molar terms) excess low molecular weight polyols are polyols having molar masses of 62 to 299 daltons, having 2 to 12 carbon atoms and hydroxyl functionalities of at least 2, which may further be branched or unbranched and whose hydroxyl groups are primary or secondary. These low molecular weight polyols can also have ether groups. Typical representatives are ethylene glycol, 1,2-propanediol, 1,3-propanediol, 1,4-butanediol, 2,3-butanediol, 2-methyl-1,3-propanediol, 1,5-pentanediol, 1,6-hexanediol, 3-methyl-1,5-pentanediol, 1,8-octanediol, 1,10-decanediol, 1,12-dodecanediol, cyclohexanediol, diethylene glycol, triethylene glycol and higher homologs, dipropylene glycol, tripropylene glycol and higher homologs, glycerol, 1,1,1-trimethylolpropane and also oligotetrahydrofurans having hydroxyl end groups. It will be appreciated that mixtures can also be used within this group.

Higher molecular weight polyols excess in molar terms are polyols having molar masses of 300 to 3000 daltons, which are obtained by ring-opening polymerization of epoxides, preferably ethylene oxide, propylene oxide and/or butene oxide, and also by acid-catalyzed, ring-opening polymerization of tetrahydrofuran.

Useful polyols A2) also include for example polyesterether polyols based on ester starters. They are obtainable using double metal cyanide compounds (“DMC catalysts”) for the alkylene oxide addition onto ester-based starter compounds having Zerevitinov-active hydrogen atoms. The standard base-catalyzed addition reaction of alkylene oxides cannot be used in this case since it would cause the starter molecules to hydrolyze.

Hydrogen attached to N, O or S is known as “Zerevitinov-active” hydrogen (sometimes also just “active hydrogen”) if, in accordance with a method found by Zerevitinov, it reacts with methyl magnesium iodide to provide methane. Typical examples of compounds having Zerevitinov-active hydrogen are compounds that contain carboxyl, hydroxyl, amino, imino or thiol groups as functional groups.

The use of high-activity DMC catalysts, described for example in U.S. Pat. No. 5,470,813, EP-A 700 949, EP-A 743 093, EP-A 761 708, WO 97/40086, WO 98/16310 and WO 00/47649, enables polyesterether polyol production at very low catalyst concentrations (25 ppm or less), so that it is no longer necessary to remove the catalyst from the final product. DMC catalysis, furthermore, enables production of polyesterether polyols based on propylene oxide or on propylene oxide-ethylene oxide mixed-block structures having very high molar masses.

In general, the starter molecules initially charged to an autoclave are reacted with alkylene oxides under inert gas at temperatures of 60-180° C., preferably at 100-170° C. in the presence of the alkylene oxide addition catalyst by continuously feeding the alkylene oxides into the reactor in the usual manner so as not to exceed the safe pressure limits of the reactor system used. It is advisable to precede the alkylene oxide metering phase with an additional stripping step with inert gases in order that any trace amounts of water or other low molecular weight impurities that interfere with DMC catalysis may be removed from the starting medium.

The reactions are typically carried out in the pressure range from 10 mbar to 10 bar. Completion of the alkylene oxide metering phase is followed by a secondary reaction phase during which the remaining alkylene oxide abreacts. This secondary reaction phase ends once there is no further detectable pressure decrease in the reaction tank. To completely remove unconverted epoxides, the secondary reaction phase can be followed by a vacuum or stripping step with inert gases or water vapor.

Useful alkylene oxides include for example ethylene oxide, propylene oxide, 1,2-butylene oxide, 2,3-butylene oxide, styrene oxide, 1,2-dodecene oxide and respectively glycidyl ester and glycidyl ether derivatives. Propylene oxide, ethylene oxide and 1,2-butylene oxide are preferably used. The various alkylene oxides can be dosed in admixture or blockwise. Products having ethylene oxide end blocks are characterized for example by elevated concentrations of primary end groups, which endow the systems with an elevated isocyanate reactivity. Preferred products are prepared using ethylene oxide in amounts >50 wt % and more preferably >60 wt %, based on the total amount of dosed epoxides.

Suitable starter molecules containing Zerevitinov-active hydrogen atoms have functionalities in the range from 2 to 4. They are prepared similarly to the polyester polyols, as described in EP 2 095 832 A1), from hydroxyl- or amino-functional low molecular weight compounds, by esterification.

Examples of hydroxyl-functional starter molecules are propylene glycol, ethylene glycol, diethylene glycol, dipropylene glycol, 1,2-butanediol, 1,3-butanediol, 1,4-butanediol, hexanediol, pentanediol, 3-methyl-1,5-pentanediol, 1,12-dodecanediol, glycerol, trimethylolpropane, triethanolamine, pentaerythritol, hydroquinone, pyrocatechol, resorcinol, bisphenol F, bisphenol A and 1,3,5-trihydroxybenzene. Examples of amino-functional starter molecules are ammonia, ethanolamine, diethanolamine, isopropanolamine, diisopropanolamine, ethylenediamine, hexamethylenediamine, aniline, the isomers of toluidine, the isomers of diaminotoluene and the isomers of diaminodiphenylmethane. Useful starter molecules also include ring-opening products from cyclic carboxylic anhydrides and polyols. Examples are ring-opening products from phthalic anhydride, succinic anhydride, maleic anhydride on the one hand and ethylene glycol, diethylene glycol, 1,2-butanediol, 1,3-butanediol, 1,4-butanediol, hexanediol, pentanediol, 3-methyl-1,5-pentanediol, 1,12-dodecanediol, glycerol, trimethylolpropane or pentaerythritol on the other. It will be appreciated that mixtures of various starter molecules can also be used.

Starter molecules have OH numbers <400 mg KOH/g and preferably <300 mg KOH/g.

Polyetherester polyols are alternatively also obtainable directly using DMC catalysis via ring-opening copolymerization of alkylene oxides and lactones/cyclic dicarboxylic anhydrides (such as for example phthalic anhydride, succinic anhydride, etc.) onto polyfunctional starter molecules. Suitable processes resemble those described above for the DMC-catalyzed preparation of polyetherester polyols in that, as well as the alkylene oxides, suitable lactones and/or cyclic dicarboxylic anhydrides are simply co-dosed as additional monomers. Reference may be made in this connection to DE 17 70 548 A, U.S. Pat. No. 5,145,883 and U.S. Pat. No. 5,032,671.

Suitable polyesterether polyols have a hydroxyl number of 5 to 140 mg KOH/g and preferably of 20 to 130 mg KOH/g.

The polyether polyols optionally used in A2) as a blending component have a molecular weight in the range from 100 to 2000 g/mol, preferably in the range from 100 to 1000 g/mol and more preferably in the range from 100 to 400 g/mol. Their polyether chains consist wholly or partly of polyethylene oxide units.

When A2) utilizes polyether polyols alongside the polyesters or polyetheresters, their proportion will comprise not more than 70% by weight and preferably not more than 50% by weight based on the entire component A2).

Preferably the mass fraction of the entire component A2) that is attributable to ethylene oxide is preferably in the range from 40% to 95% by weight and more preferably in the range from 60% to 90% by weight.

Component A2) preferably has an ester group concentration (in moles per kg) of 0.5 to 5.5 and more preferably 1 to 3.5.

Component A2) may further also have carbonate structural units. Depending on the type of polyols used for carbonate formation, different types of carbonate polyols are obtained: When oligoester polyols are carbonated for example, polyestercarbonate polyols are obtained. When the oligoesters in turn contain for example ether groups, e.g., from oligoethylene glycols such as diethylene glycol for example, then polyetherestercarbonate polyols are obtained, and so on.

The carbonation reaction is known per se to a person skilled in the art. Useful sources of carbonyl include especially diphenyl carbonate, dimethyl carbonate, but also phosgene or chlorocarbonic esters. Diphenyl carbonate (DPC) and dimethyl carbonate are preferable and diphenyl carbonate (DPC) is very particularly preferable.

Polyisocyanates A1) may preferably have an average NCO functionality in the range from 2 to 2.6 and more preferably in the range from 2 to 2.4.

Polyisocyanates A1) may be monomeric aliphatic and/or cycloaliphatic di- or triisocyanates, especially 1,4-butylene diisocyanate (BDI), 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes and/or their mixtures of any desired isomer content, 1,4-cyclohexylene diisocyanate, 4-isocyanatomethyl-1,8-octane diisocyanate (nonane triisocyanate), and/or alkyl 2,6-diisocyanatohexanoate (lysine diisocyanate) with C1-C8 alkyl groups and/or mixtures of the foregoing polyisocyanates.

Hexamethylene diisocyanate is very particularly preferable.

Polyisocyanate prepolymers A) preferably contain less than 0.5 wt % and more preferably less than 0.03 wt % of monomeric di- and/or triisocyanate. This can be realized for example by preparing the polyisocyanate prepolymers in the presence of an excess of di- and/or triisocyanate and then removing unconverted di- and/or triisocyanate using thin film distillation.

It is further preferable for polyisocyanate prepolymers A) to have an NCO functionality of 2 to 6 and preferably of 3 to 4.

In principle, prepolymer preparation may also utilize known catalysts per se such as amines or tin compounds and also stabilizers such as benzoyl chloride, isophthaloyl chloride, dibutyl phosphate or methyl tosylate.

Polyisocyanate prepolymers A) preferably have a miscibility with water at 25° C. of at least 2 wt % based on the resulting mixture. It is particularly preferable for them to form a homogeneous and clear mixture with water at 25° C. in any proportion.

Examples of hydroxyl-amino compounds C) are aminoalcohols such as triethanolamine or tripropanolamine or ammonia-, di/polyamine- or aminoalcohol-initiated polyalkylene oxides where, for example, ethylene oxide, propylene oxide, but also butylene oxide or styrene oxide can be used singly, in admixture or for blockwise construction.

The hydrogels are prepared using water B) in such amounts that gel formation is achieved, which in the individual case is experimentally determined in preliminary tests. It is preferable to use from 2 to 50 parts by weight and more preferably from 4 to 19 parts by weight of water based on the weight quantity of the compounds used in a) and b) (corresponding to one part by weight).

An optional step in the hydrogel-preparing process comprises mixing water B) with hydroxy-amino compounds C), in which case the said hydroxy-amino compounds C) are used in amounts of 0.1-5 wt % and preferably of 0.1-1% on the total amount of A) and C). The mixture is then added to polyisocyanate prepolymers A) and stirred in until a clear solution has formed. Stirring is typically done at room temperature, but can also be done at temperatures above room temperature at temperatures of 23 to 40 or else at temperatures of 30 to 80° C. The temperature may further be below room temperature, for example in the range from 5 to 23° C. or else from −10 to +10° C.

A magnetic stirrer with a cross stirbar will be found advantageous as stirring assembly, but a speedmixer or a customary laboratory blade or grid stirrer can also be used. The choice of mixing assembly in the individual case depends for example on the quantity to be stirred and on its viscosity.

Stirring can also be done in a protective gas atmosphere, for example under nitrogen. Normally, a protective gas atmosphere is not used. Furthermore, mixing can take place under atmospheric pressure. But it is also possible for stirring to take place under slightly elevated pressure, for example at 1013 to 1035 mbar or else under reduced pressure for example at 800 to 1013 mbar.

To improve visibility of the resultant gel on the tissue, the hydrogel can be stained. Methylene blue or the food dye Brilliant Blue FCF is suitable for this for example. The dye is preferably added to water B).

It will be appreciated that pharmacologically active ingredients such as, for example,
a) anti-inflammatories,
b) analgesics with and without anti-inflammatory effect,
c) antimicrobially active substances,
d) vasodilators,
e) growth factors

can also be incorporated.

Polyisocyanate prepolymers A) have a DIN EN ISO 11909 average NCO content of 2 to 10 wt % and preferably of 2.5 to 8 wt %.

The invention further provides a process for preparing a hydrogel, which process comprises
i) reacting polyisocyanates with polyols having hydrolyzable groups in the polymer chain to form polyisocyanate prepolymers, and
ii) optionally mixing water with compounds having at least one tertiary amino group and at least three hydroxyl groups,
iii) adding the mixture of step ii) to the prepolymers of step i) and stirring.

The invention also provides a hydrogel obtainable via the process.

The invention likewise provides a method of using the hydrogels as an adhesion barrier and also their use as coatings for sealing, uniting or covering cell tissues, while cell tissue can be not only human cell tissue but also animal cell tissue.

When the hydrogel is to be used as an adhesion barrier, it can be sensible to color one or more of components A) to C) used to make the barrier easier to see.

In the in vivo application of a coating to produce a postoperative adhesion barrier, the necessary components are applied, with the aid of a two-chamber dispensing system and a suitable applicator, to the organ to be protected. One chamber contains isocyanate prepolymer A, the second chamber contains water (B), optionally mixed with the hydroxyamino compound C, and also D and E. When pharmacologically active substances are used, these are formulated in the aqueous component. The hydrogel forms a protective polymeric film on the organ. This film adheres to the organ surface without penetrating into the tissue. The film can be mechanically removed without damaging the tissue.

### EXAMPLES

Apparatus and Analytical Methods Used:
viscometer: MCR 51, Anton Paar, determination to DIN EN ISO 3219/A.3
hydroxyl number: determination to DIN 53240
acid number: determination to DIN 53402

Raw Materials Used:
Polyether L5050: bifunctionally initiated EO-PO polyether, Bayer MaterialScience AG, with a hydroxyl number of about 57 mg KOH/g.
Polyether L300: bifunctionally initiated EO polyether, Bayer MaterialScience AG, with a hydroxyl number of about 190 mg KOH/g.

Desmophen
VP.PU 41WB01: trifunctionally initiated polyether, Bayer MaterialScience AG, with a hydroxyl number of about 37 mg KOH/g.
Polyether V657: trifunctionally initiated polyether, Bayer MaterialScience AG, with a hydroxyl number of about 255 mg KOH/g.
ε-caprolactone: Perstorp
HDI (hexamethylene diisocyanate): Bayer MaterialScience AG
benzoyl chloride: Aldrich
adipic acid: BASF
pentaerythritol: Aldrich
tin dichloride dihydrate: Aldrich
ethylene oxide: Gerling, Holz & Co
butylene oxide: Aldrich
propylene oxide: Chemogas
trimethylolpropane: Aldrich
Irganox 1076: Ciba
dibutyl phosphate: Aldrich
diphenyl carbonate: Bayer MaterialScience AG
DMC catalyst: double metal cyanide catalyst containing zinc hexacyanocobaltate, tert-butanol and polypropylene glycol with a number average molecular weight of 1000 g/mol; described in EP-A 700 949

Synthesis of Polyesterether Prepolymers

### Example 1

A 4-liter 4-neck flask equipped with heating mantle, mechanical stirrer, internal thermometer and reflux condenser is initially charged with 762 g (5.6 mol) of pentaerythritol, 2554 g (22.4 mol) of ε-caprolactone and 66 mg (20 ppm) of tin dichloride dihydrate at 100° C. under nitrogen blanketing. The temperature is raised to 200° C. in the course of 1 hour and the reaction is completed under these conditions for a further 20 hours. The compound obtained has the following properties:

hydroxyl number: 373 mg KOH/g

acid number: 0.5 mg KOH/g

viscosity: 190 mPas (75° C.)

### Example 2

A 2-liter stainless steel pressure reactor is initially charged with 179.3 g of compound from example 1 and also 0.52 g of DMC catalyst (prepared as described in EP-A 700 949) under nitrogen. The initial charge was then heated to 130° C. After 1 h of stripping with nitrogen at 0.1 bar, the metered addition is commenced at 130° C. of ethylene oxide and butylene oxide in a weight ratio of 75/25. After 618 g of ethylene oxide and 206 g of butylene oxide have been added in the course of 2 h, metering is interrupted and 425.5 g of product are removed from the reactor. Then, a further 694 g of ethylene oxide and 231 g of butylene oxide are added at 130° C. in the course of 2 h. Following a secondary reaction period of 45 min at 130° C., volatiles are distilled off in vacuo at 130° C. for 30 min and the reaction mixture is subsequently cooled down to room temperature.

Product Properties:

OH number: 25.5 mg KOH/g

viscosity (25° C.): 5780 mPas

### Example 3

### Prepolymer 3

276 g of HDI and 1 g of benzoyl chloride are initially charged to a 1 l four-neck flask. In the course of 2 h, 724 g of compound from example 2 are added and subsequently stirred for 1 h, at 80° C. Excess HDI is then distilled off by thin film distillation at 130° C. and 0.13 mbar to obtain prepolymer 1 with an NCO content of 1.54 wt %. The residual monomer content determined to DIN EN ISO 10283 was <0.03 wt % of HDI. Viscosity: 15 600 mPas (23° C.).

### Example 4

A 4-liter 4-neck flask equipped with heating mantle, mechanical stirrer, internal thermometer and reflux condenser is initially charged with 911 g (6.8 mol) of 1,1,1-trimethylolpropane, 2326 g (20.4 mol) of ε-caprolactone and 64 mg (20 ppm) of tin dichloride dihydrate at 100° C. under nitrogen blanketing. The temperature is raised to 200° C. in the course of 1 hour and the reaction is completed under these conditions for a further 20 hours. The compound obtained has the following properties:

hydroxyl number: 346 mg KOH/g

acid number: 0.2 mg KOH/g

viscosity: 1510 mPas (25° C.), 100 mPas (75° C.)

### Example 5

A 2-liter stainless steel pressure reactor is initially charged with 175.5 g of compound from example 4 and also 0.48 g of DMC catalyst (prepared as described in EP-A 700 949) under nitrogen. The initial charge was then heated to 130° C. After 1 h of stripping with nitrogen at 0.1 bar, the metered addition is commenced at 130° C. of ethylene oxide and butylene oxide in a weight ratio of 75/25. After 618 g of ethylene oxide and 206 g of butylene oxide have been added in the course of 2 h, metering is interrupted and 382.5 g of product are removed from the reactor. Then, a further 662 g of ethylene oxide and 221 g of butylene oxide are added at 130° C. in the course of 2 h. Following a secondary reaction period of 45 min at 130° C., volatiles are distilled off in vacuo at 130° C. for 30 min and the reaction mixture is subsequently cooled down to room temperature.

Product Properties:

hydroxyl number: 25.1 mg KOH/g

viscosity (25° C.): 3170 mPas

### Example 6

### Prepolymer 6

273 g of HDI and 1 g of benzoyl chloride are initially charged to a 1 l four-neck flask. In the course of 2 h, 727 g of precursor from example 5 are added and subsequently stirred for 1 h, at 80° C. Excess HDI is then distilled off by thin film distillation at 130° C. and 0.13 mbar to obtain prepolymer 6 with an NCO content of 1.7 wt %. The residual monomer content (determined to DIN EN ISO 10283) was <0.03 wt % of HDI. Viscosity: 12 200 mPas (23° C.).

### Example 7

In a 2-liter stainless steel pressure reactor 198.2 g of a trifunctional polyether starter molecule (construction: glycerol←PO/EO (40/60); OH number=260 mg KOH/g) and also 0.12 g of DMC catalyst (prepared as described in EP-A 700 949) are initially charged, and then heated to 130° C., under nitrogen. After 1 h of stripping with nitrogen at 0.1 bar, the metered addition of ethylene oxide, propylene oxide and caprolactone is commenced at 130° C. After initially 561 g of ethylene oxide, 160 g of propylene oxide and 100 g of ε-caprolactone have been added in the course of 2.5 h, the metering of caprolactone is interrupted and then, at 130° C., a further 140 g of ethylene oxide and 40 g of propylene oxide are added in the course of 0.5 h. The weight ratio of the monomers added is thus: ethylene oxide/propylene oxide/ε-caprolactone=70/20/10. Following a secondary reaction period of 2 h at 130° C., volatiles are distilled off in vacuo at 130° C. for 30 min and the reaction mixture is subsequently cooled down to room temperature.

Product Properties:

hydroxyl number: 36.6 mg KOH/g

viscosity (25° C.): 1427 mPas

### Example 8

### Prepolymer 8

732.4 g of HDI and 3.7 g of benzoyl chloride are initially charged to a 3 l four-neck flask. In the course of 2 h, 1532 g of precursor from example 7 are added and subsequently stirred for 1 h, at 80° C. Excess HDI is then distilled off by thin film distillation at 130° C. and 0.13 mbar to obtain prepolymer 8 with an NCO content of 2.47 wt %. The residual monomer content (GC) was 0.06 wt % of HDI.

### Example 9

In a 2-liter stainless steel pressure reactor 201.4 g of a trifunctional polyether starter molecule (construction: glycerol F PO/EO (30/70); OH number=37.0 mg KOH/g) and also 0.32 g of DMC catalyst (prepared as described in EP-A 700 949) are initially charged, and then heated to 130° C., under nitrogen. After 1 h of stripping with nitrogen at 0.1 bar, the metered addition of ethylene oxide, propylene oxide, ε-caprolactone and glycerol is commenced at 130° C. After initially 768 g of ethylene oxide, 219 g of propylene oxide, 137 g of ε-caprolactone and 28 g of glycerol had been added in the course of 3 h, the ε-caprolactone and glycerol metering was interrupted and then a further 165 g of ethylene oxide and 33 g of propylene oxide were added at 130° C. in the course of 0.5 h. Following a secondary reaction period of 30 min at 130° C., volatiles are distilled off in vacuo at 130° C. for 30 min and the reaction mixture is subsequently cooled down to room temperature.

Product Properties:

hydroxyl number: 34.5 mg KOH/g

viscosity (25° C.): 2513 mPas

### Example 10

### Prepolymer 10

85.17 g of HDI and 0.25 g of benzoyl chloride are initially charged to a 1 l four-neck flask. In the course of 2 h, 164.58 g of precursor from example 9 are added and subsequently stirred for 1 h, at 80° C. Excess HDI is then distilled off by thin film distillation at 130° C. and 0.13 mbar to obtain prepolymer 10 with an NCO content of 1.89 wt %. The residual monomer content (GC) was <0.03 wt % of HDI.

### Example 11

A 4-liter four-neck flask equipped with heating mantle, mechanical stirrer, internal thermometer and reflux condenser is initially charged with 1650 g (2.5 mol) of Polyether V657, 570 g (5 mol) of ε-caprolactone and 45 mg (20 ppm) of tin dichloride dihydrate at 100° C. under nitrogen blanketing. The temperature is raised to 200° C. in the course of 1 hour and the reaction is completed under these conditions for a further 20 hours. The compound obtained has the following properties:

hydroxyl number: 191 mg KOH/g

acid number: 0.5 mg KOH/g

viscosity: 430 mPas (25° C.)

### Example 12

A 4-liter 4-neck flask equipped with heating mantle, mechanical stirrer, internal thermometer and reflux condenser is initially charged with 664 g (7 mol) of glycerol, 1596 g (14 mol) of ε-caprolactone and 45 mg (20 ppm) of tin dichloride dihydrate at 100° C. under nitrogen blanketing. The temperature is raised to 200° C. in the course of 1 hour and the reaction is completed under these conditions for a further 20 hours. The compound obtained has the following properties:

hydroxyl number: 493 mg KOH/g

acid number: 0.2 mg KOH/g

viscosity: 240 mPas (50° C.), 80 mPas (75° C.)

### Example 13

A 20-liter stainless steel pressure reactor is initially charged with 1800 g of precursor from example 11 and also 0.9 g of DMC catalyst (prepared as described in EP-A 700 949) under nitrogen. The initial charge was then heated to 130° C. After 1 h of stripping with nitrogen at 0.1 bar, the metered addition is commenced at 130° C. of ethylene oxide and propylene oxide in a weight ratio of 69/31. After 5088 g of ethylene oxide and 2309 g of propylene oxide have been added in the course of 3 h, following a secondary reaction period of 60 min at 130° C., volatiles are distilled off in vacuo for 30 min and the reaction mixture is subsequently cooled down to room temperature.

Product Properties:

hydroxyl number: 37.4 mg KOH/g

viscosity (25° C.): 1275 mPas

### Example 14

A 20-liter stainless steel pressure reactor is initially charged with 1566 g of precursor from example 12 and also 1.0 g of DMC catalyst (prepared as described in EP-A 700 949) under nitrogen. The initial charge was then heated to 130° C. After 1 h of stripping with nitrogen at 0.1 bar, the metered addition is commenced at 130° C. of ethylene oxide and propylene oxide in a weight ratio of 69/31. After 8484 g of ethylene oxide and 3748 g of propylene oxide have been added in the course of 3 h, following a secondary reaction period of 60 min at 130° C., volatiles are distilled off in vacuo for 30 min and the reaction mixture is subsequently cooled down to room temperature.

Product Properties:

hydroxyl number: 55.2 mg KOH/g

viscosity (25° C.): 944 mPas

### Example 15

A 20-liter stainless steel pressure reactor is initially charged with 1403 g of precursor from example 1 and also 4.8 g of DMC catalyst (prepared as described in EP-A 700 949) under nitrogen. The initial charge was then heated to 130° C. After 1 h of stripping with nitrogen at 0.1 bar, the metered addition is commenced at 130° C. of ethylene oxide and propylene oxide. After 9124 g of ethylene oxide and 2603 g of propylene oxide have been added in the course of 3 h, metering is interrupted and, following a secondary reaction period of 60 min, 8436 g of product are removed from the reactor. Then, a further 1498 g of ethylene oxide and 642 g of propylene oxide are added at 130° C. in the course of 3 h (addition in 2 blocks has merely technical reasons: Owing to the large OH number difference between the starter molecule and the end product, the amount of starter molecule to be used for a one-step addition is too small for the type of reactor used. Following a secondary reaction period of 60 min at 130° C., volatiles are distilled off in vacuo for 30 min and the reaction mixture is subsequently cooled down to room temperature.

Product Properties:

hydroxyl number: 24.7 mg KOH/g

viscosity (25° C.): 4403 mPas

### Example 16

A 20-liter stainless steel pressure reactor is initially charged with 1436 g of precursor from example 4 and also 4.8 g of DMC catalyst (prepared as described in EP-A 700 949) under nitrogen. The initial charge was then heated to 130° C. After 1 h of stripping with nitrogen at 0.1 bar, the metered addition is commenced at 130° C. of ethylene oxide and propylene oxide. After 9310 g of ethylene oxide and 2553 g of propylene oxide have been added in the course of 3 h, metering is interrupted and, following a secondary reaction period of 60 min, 9506 g of product are removed from the reactor. Then, a further 1338 g of ethylene oxide and 577 g of propylene oxide are added at 130° C. in the course of 3 h (addition in 2 blocks has merely technical reasons: Owing to the large OH number difference between the starter molecule and the end product, the amount of starter molecule to be used for a one-step addition is too small for the type of reactor used). Following a secondary reaction period of 60 min at 130° C., volatiles are distilled off in vacuo for 30 min and the reaction mixture is subsequently cooled down to room temperature.

Product Properties:

hydroxyl number: 24.5 mg KOH/g

viscosity (25° C.): 3806 mPas

### Example 17

### Prepolymer 17

359 g of HDI and 1 g of benzoyl chloride are initially charged to a 2 l four-neck flask. In the course of 2 h, 641 g of precursor from example 13 are added and subsequently stirred for 1 h, at 80° C. Excess HDI is then distilled off by thin film distillation at 130° C. and 0.13 mbar to obtain prepolymer 17 with an NCO content of 2.27 wt % and a viscosity of 4570 mPas (23° C.). The residual monomer content was <0.03 wt % of HDI.

### Example 18

### Prepolymer 18

453 g of HDI and 1 g of benzoyl chloride are initially charged to a 2 1 four-neck flask. In the course of 2 h, 547 g of precursor from example 14 are added and subsequently stirred for 1 h, at 80° C. Excess HDI is then distilled off by thin film distillation at 130° C. and 0.13 mbar to obtain prepolymer 18 with an NCO content of 3.32 wt % and a viscosity of 3430 mPas (23° C.). The residual monomer content was <0.03 wt % of HDI.

### Example 19

### Prepolymer 19

270 g of HDI and 1 g of benzoyl chloride are initially charged to a 2 l four-neck flask. In the course of 2 h, 730 g of precursor from example 15 are added and subsequently stirred for 1 h, at 80° C. Excess HDI is then distilled off by thin film distillation at 130° C. and 0.13 mbar to obtain prepolymer 19 with an NCO content of 1.66 wt % and a viscosity of 20 200 mPas (23° C.). The residual monomer content was <0.03 wt % of HDI.

### Example 20

### Prepolymer 20

360 g of HDI and 1 g of benzoyl chloride are initially charged to a 2 l four-neck flask. In the course of 2 h, 640 g of precursor from example 16 are added and subsequently stirred for 1 h, at 80° C. Excess HDI is then distilled off by thin film distillation at 130° C. and 0.1 Torr to obtain prepolymer 20 with an NCO content of 2.3 wt % and a viscosity of 5960 mPas (23° C.). The residual monomer content was <0.03 wt % of HDI.

### Example 21

A 4-liter four-neck flask equipped with heating mantle, mechanical stirrer, internal thermometer, 40 cm packed column, column head, descending intensive condenser and also membrane vacuum pump is initially charged with weighed-out 1152 g (1.95 mol) of Polyether L300, 1535 g (0.34 mol) of Desmophen VP.PU 41WB01, 98 g (0.73 mol) of 1,1,1-trimethylolpropane and 285 g (1.95 mol) of adipic acid under nitrogen blanketing. The initial charge is heated to 200° C. under atmospheric pressure while water distills off. After 4 hours 60 mg (corresponding to 20 ppm) of tin dichloride dihydrate are added under nitrogen blanketing. The pressure is reduced in the course of 1 hour to finally 15 mbar and the reaction is completed under these conditions for a further 48 hours. The product has the following properties:

hydroxyl number: 57 mg KOH/g

acid number: 1.1 mg KOH/g

viscosity: 4580 mPas (25° C.), 1310 mPas (50° C.), 570 mPas (75° C.)

### Example 22

### Prepolymer 22

101.43 g of HDI and 0.28 g of benzoyl chloride are initially charged to a 1 l four-neck flask. In the course of 2 h, 148.29 g of precursor from example 21 are added and subsequently stirred for 1 h, at 80° C. Excess HDI is then distilled off by thin film distillation at 130° C. and 0.13 mbar to obtain prepolymer 22 with an NCO content of 3.37 wt %. The residual monomer content was <0.03 wt % of HDI.

### Example 23

A 4-liter four-neck flask equipped with heating mantle, mechanical stirrer, internal thermometer, 40 cm packed column, column head, descending intensive condenser and also membrane vacuum pump is initially charged with weighed-out 1078 g (1.82 mol) of Polyether L300, 1533 g (0.34 mol) of Desmophen VP.PU 41WB01, 146 g (1.09 mol) of 1,1,1-trimethylolpropane, 155 g (1.06 mol) of adipic acid and 1.55 g (0.77 mol) of sebacic acid under nitrogen blanketing. The initial charge is heated to 200° C. under atmospheric pressure while water distills off. After 4 hours 60 mg (corresponding to 20 ppm) of tin dichloride dihydrate are added under nitrogen blanketing. The pressure is reduced in the course of 1 hour to finally 15 mbar and the reaction is completed under these conditions for a further 48 hours. After cooling to 80° C., 300 mg (100 ppm) of dibutyl phosphate are stirred in. The product has the following properties:

hydroxyl number: 76 mg KOH/g

acid number: 0.9 mg KOH/g

viscosity: 2710 mPas (25° C.), 790 mPas (50° C.), 350 mPas (75° C.)

### Example 24

### Prepolymer 24

132.96 g of HDI and 0.25 g of benzoyl chloride are initially charged to a 1 l four-neck flask. In the course of 2 h, 116.79 g of precursor from example 23 are added and subsequently stirred for 1 h, at 80° C. Excess HDI is then distilled off by thin film distillation at 130° C. and 0.13 mbar to obtain prepolymer 24 with an NCO content of 4.27 wt %. The residual monomer content was <0.03 wt % of HDI.

### Example 25

A 4-liter four-neck flask equipped with heating mantle, mechanical stirrer, internal thermometer, 40 cm packed column, column head, descending intensive condenser and also membrane vacuum pump is initially charged with weighed-out 1894 g (0.95 mol) of Polyether L5050, 341 g (0.58 mol) of Polyether L300, 248 g (1.24 mol) of polyethylene glycol 300, 213 g (2.32 mol) of glycerol, 403 g (2.76 mol) of adipic acid and 883 g (7.75 mol) of ε-caprolactone under nitrogen blanketing. The initial charge is heated to 200° C. under atmospheric pressure while water distills off. After 4 hours 60 mg (20 ppm) of tin dichloride dihydrate are added under nitrogen blanketing. The pressure is reduced in the course of 1 hour to finally 15 mbar and the reaction is completed under these conditions for a further 48 hours. After cooling to 80° C., 300 mg (100 ppm) of dibutyl phosphate are stirred in. The product has the following properties:

hydroxyl number: 92 mg KOH/g

acid number: 0.3 mg KOH/g

viscosity: 2470 mPas (25° C.), 640 mPas (50° C.), 260 mPas (75° C.)

### Example 26

### Prepolymer 26

173.46 g of HDI and 0.3 g of benzoyl chloride are initially charged to a 1 l four-neck flask. In the course of 2 h, 126.24 g of precursor from example 25 are added and subsequently stirred for 1 h, at 80° C. Excess HDI is then distilled off by thin film distillation at 130° C. and 0.13 mbar to obtain prepolymer 26 with an NCO content of 4.71 wt %. The residual monomer content was <0.03 wt % of HDI.

### Example 27

A 10-liter four-neck flask equipped with heating mantle, mechanical stirrer, internal thermometer, 40 cm packed column, heatable distillation bridge, heatable descending intensive condenser, and also membrane vacuum pump and oil pump is initially charged with weighed-out 375 g (2.50 mol) of triethylene glycol, 4663 g (1.03 mol) of Polyether VP.PU 41WB01, 385 g (3.38 mol) of ε-caprolactone and 75 mg of dibutyltin oxide, and the initial charge is stirred at 200° C. under nitrogen for 20 hours. After cooling to 150° C., 530 g (2.65 mol) of polyethylene glycol 200, 355 g (2.65 mol) of 1,1,1-trimethylolpropane, 1103 g (5.15 mol) of diphenyl carbonate and 75 mg of dibutyltin oxide are added. This is followed by stirring at 180° C. under nitrogen at atmospheric pressure for 1 hour, cooling to 120° C., pressure reduction to 15 mbar and heating of the bridge and condenser with hot water at 45° C., while phenol distills off. The temperature is increased to 200° C. in the course of 10 hours, during which 871 g of phenol distill off. The pressure is reduced to 1 mbar using the oil pump and the reaction is completed in the course of 2 hours, during which a further 107 g of phenol distill off After cooling to 80° C. 640 mg (100 ppm) of dibutyl phosphate are stirred in. The product has the following properties:

hydroxyl number: 89 mg KOH/g

acid number: 0.2 mg KOH/g

viscosity: 2690 mPas (25° C.), 740 mPas (50° C.), 310 mPas (75° C.)

free phenol: 0.02 wt % (GC)

### Example 28

### Prepolymer 28

142.58 g of HDI and 0.25 g of benzoyl chloride are initially charged to a 1 l four-neck flask. In the course of 2 h, 107.16 g of precursor from example 27 are added and subsequently stirred for 1 h, at 80° C. Excess HDI is then distilled off by thin film distillation at 130° C. and 0.1 Torr to obtain prepolymer 28 with an NCO content of 4.92 wt %. The residual monomer content was <0.03 wt % of HDI.

### Example 29

A 2-liter four-neck flask equipped with heating mantle, mechanical stirrer, internal thermometer, 40 cm packed column, column head, descending intensive condenser and also membrane vacuum pump is initially charged with weighed-out 141.7 g (1.2 mol) of succinic acid, 720 g (1.2 mol) of polyethylene glycol 600 and 25.4 g (0.27 mol) of glycerol under nitrogen blanketing. The initial charge is heated to 200° C. under atmospheric pressure while water distills off. After 4 hours 89 mg (100 ppm) of tin dichloride dihydrate are added under nitrogen blanketing. The pressure is reduced in the course of 1 hour to finally 15 mbar and the reaction is completed under these conditions for a further 48 hours. After cooling to 80° C., 300 mg (100 ppm) of dibutyl phosphate are stirred in. The product has the following properties:

hydroxyl number: 46 mg KOH/g

acid number: 0.6 mg KOH/g

### Example 30

### Prepolymer 30

252 g of HDI and 0.62 g of benzoyl chloride are initially charged to a 21 four-neck flask.

In the course of 2 h, 365.2 g of precursor from example 29 are added and subsequently stirred for 1 h, at 80° C. Excess HDI is then distilled off by thin film distillation at 130° C. and 0.13 mbar to obtain prepolymer 30 with an NCO content of 3.1 wt %. The residual monomer content was 0.09 wt % of HDI, the viscosity was 22 400 mPas (25° C.).

### Example 31

A 2-liter four-neck flask equipped with heating mantle, mechanical stirrer, internal thermometer, 40 cm packed column, column head, descending intensive condenser and also membrane vacuum pump is initially charged with weighed-out 175.4 g (1.4 mol) of adipic acid, 720 g (0.6 mol) of polyethylene glycol 600 and 34.8 g (0.12 mol) of trimethylolpropane under nitrogen blanketing. The initial charge is heated to 200° C. under atmospheric pressure while water distills off After 4 hours 90 mg (100 ppm) of tin dichloride dihydrate are added under nitrogen blanketing. The pressure is reduced in the course of 1 hour to finally 15 mbar and the reaction is completed under these conditions for a further 48 hours. After cooling to 80° C., 300 mg (100 ppm) of dibutyl phosphate are stirred in. The product has the following properties:

hydroxyl number: 43 mg KOH/g

acid number: 0.2 mg KOH/g

### Example 32

### Prepolymer 32

400 g of HDI and 1.02 g of benzoyl chloride are initially charged to a 21 four-neck flask. In the course of 2 h, 621.3 g of precursor from example 31 are added and subsequently stirred for 1 h, at 80° C. Excess HDI is then distilled off by thin film distillation at 130° C. and 0.13 mbar to obtain prepolymer 32 with an NCO content of 2.99 wt %. The residual monomer content was <0.03 wt % of HDI, the viscosity was 28 000 mPas (25° C.).

Preparation of Hydrogels

The hydrogels were each prepared by stirring 1 g of the appropriate prepolymer with a mixture of 8 g of water and 0.06 g of triethanolamine using a magnetic stirrer with cross stirbar for 1 min. The (processing) time was measured for a solid gel to form.

| | Processing |
|---|---|
| Prepolymer | time [min] |
| 3 | 4 |
| 6 | 9 |
| 8 | 4 |
| 10 | 6 |
| 17 | 4 |
| 18 | 8 |
| 19 | 3 |
| 20 | 10 |
| 22 | 5 |
| 24 | 5 |
| 26 | 7 |
| 28 | 3 |
| 30 | 4 |
| 32 | 9 |

### Example 33

### Biodegradation of Hydrogels

The corresponding hydrogels were made to cure in a tube (diameter 0.5 cm, length 2 cm). The resulting test specimens 2.7 g in weight were each allowed to swell in 10 ml of buffer solution (pH 7.4, Aldrich P-5368) at 60° C. in a shaking incubator at 150 rpm for 48 h. Subsequently, the samples were rinsed off with completely ion-free water and dabbed dry. The weight of the samples was recorded as starting weight. The samples were further shaken in 10 ml of buffer solution (pH 7.4, Aldrich P-5368) at 60° C. and/or 37° C. in a shaking incubator under the same conditions. The weight of the samples was determined on a weekly basis. The hydrogel was deemed to have degraded when it had completely dissolved without leaving a sediment.

The samples were completely degraded after the following periods:

gel from 30: 7 days (60° C.), 14 days (37° C.)

gel from 20: 35 days (60° C.)

gel from 18: 42 days (60° C.)

gel from 32: 7 days (60° C.), 14 days (37° C.)

1.-9. (canceled)

10. A hydrogel based on polyurethane or polyurethaneurea, having hydrolyzable functional groups in the polymer chain and obtained by reaction of
A) a polyisocyanate prepolymer having hydrolyzable groups in the polymer chain,
B) water
C) optionally hydroxyl-amino compounds having at least one tertiary amino group and at least three hydroxyl groups,
D) optionally catalysts, and
E) optionally auxiliary and addition agents,
wherein said polyisocyanate prepolymer A) is obtained by reaction of
A1) a polyisocyanate with
A2) a polyol having hydrolyzable groups in the polymer chain,
wherein said polyol A2) comprises polyesters and/or polyetheresters that are liquid at room temperature and have a DIN 53019 shear viscosity at 23° C. in the range of from 200 to 8000 mPas.

11. The hydrogel as claimed in claim 10, wherein said polyol A2) comprises polyesters and/or polyetheresters that are liquid at room temperature and have a DIN 53019 shear viscosity at 23° C. in the range of from 400 to 4000 mPas.

12. The hydrogel as claimed in claim 10, wherein the hydrolyzable functional groups are ester, acetal and/or carbonate groups.

13. The hydrogel as claimed in claim 10, wherein the polyetheresters and/or the polyesters have a hydroxyl number of 20 to 140 mg KOH/g and/or an acid number of 0.05 to 10 mg KOH/g.

14. The hydrogel as claimed in claim 10, wherein the polyetheresters and/or the polyesters have a hydroxyl number of 20 to 100 mg KOH/g and/or an acid number of 0.1 to 3 mg KOH/g.

15. The hydrogel as claimed in claim 10, wherein the polyetheresters and/or the polyesters have an acid number of 0.1 to 3 mg KOH/g

16. The hydrogel as claimed in claim 10, wherein said polyisocyanates A1) comprise monomeric aliphatic and/or cycloaliphatic di- or triisocyanates.

17. The hydrogel as claimed in claim 10, wherein said polyisocyanates A1) are selected from the group consisting of 1,4-butylene diisocyanate (BDI), 1,6-hexamethylene diisocyanate (HDI), isophorone diisocyanate (IPDI), 2,2,4- and 2,4,4-trimethylhexamethylene diisocyanate, isomeric bis(4,4′-isocyanatocyclohexyl)methanes and their mixtures of any desired isomer content, 1,4-cyclohexylene diisocyanate, 4-isocyanatomethyl-1,8-octane diisocyanate (nonane triisocyanate), alkyl 2,6-diisocyanatohexanoate (lysine diisocyanate) with C1-C8 alkyl groups, and mixtures of the foregoing polyisocyanates.

18. The hydrogel as claimed in claim 10, wherein said polyisocyanate prepolymers A) comprise less than 0.5 wt % of monomeric di- and/or triisocyanate.

19. The hydrogel as claimed in claim 10, wherein said polyisocyanate prepolymers A) comprise less than 0.03 wt % of monomeric di- and/or triisocyanate.

20. The hydrogel as claimed in claim 10, wherein said polyisocyanate prepolymers A) have an NCO functionality of 2 to 6.

21. The hydrogel as claimed in claim 10, wherein said polyisocyanate prepolymers A) have an NCO functionality of 3 to 4.

22. The hydrogel as claimed in claim 10, wherein said hydroxyl-amino compounds C) comprise polyalkylene oxides started on trifunctional amino alcohols.

23. An adhesion barrier comprising the hydrogel as claimed in claim 10.

24. A process for preparing the hydrogel as claimed in claim 10, comprising:
i) reacting a polyisocyanate with a polyol having hydrolyzable groups in the polymer chain to form polyisocyanate prepolymers, and
ii) optionally mixing water with compounds having at least one tertiary amino group and at least three hydroxyl groups, and
iii) adding the mixture of step ii) to the prepolymers of step i) and stirring.
